(19) United States
(12) Patent Application Publication
(10) Pub. No.: US 2024/0033282 A1
(43) Pub. Date: Feb. 1, 2024

(54) METHOD FOR IDENTIFYING AND/OR FOR OBTAINING AN ACTIVE SUBSTANCE FOR THE TREATMENT AND THERAPY OF FAMILIAL AMYOTROPHIC LATERAL SCLEROSIS, AND USE OF AN ACTIVE SUBSTANCE FOR THE TREATMENT OF THIS DISEASE

(71) Applicant: Forschungszentrum Juelich GmbH, Juelich (DE)

(72) Inventor: Gultekin TAMGUNEY, Duesseldorf (DE)

(21) Appl. No.: 18/256,926

(22) PCT Filed: Nov. 5, 2021
(86) PCT No.: PCT/DE2021/000183

§ 371 Date: Jun. 12, 2023

(30) Foreign Application Priority Data
Dec. 16, 2020 (DE) 10 2020 007 691.2

(51) Int. Cl. A61K 31/727; G01N 33/50; A61K 31/737; A61P 25/28

(52) U.S. Cl. CPC A61K 31/727; G01N 33/5058; G01N 33/5038; A61K 31/737; A61P 25/28

(57) ABSTRACT

A method for identifying and/or for obtaining active substances for the alleviation or treatment of ALS includes: a. providing a composition containing cells having the cell surface receptors syndecan-3 and/or syndecan-1, b. contacting the composition from step a) with SOD1 aggregates, c. contacting the composition from step a) with the test substance to be screened, it being possible for step b) and step c) to be performed simultaneously or for step c) to be performed before step b), and d. determining the uptake of the SOD1 aggregates into the cells, and a kit for carrying out the method, are provided. Medicaments comprising pentosan polysulfate sodium (PPS) or compounds from the group of heparins or heparin-like compounds, and methods of using the same, are also provided.

### CROSS REFERENCE TO PRIOR APPLICATIONS

This application is a U.S. National Phase application under 35 U.S.C. § 371 of International Application No. PCT/DE2021/000183, filed on Nov. 5, 2021, and claims benefit to German Patent Application No. 10 2020 007 691.2, filed on Dec. 16, 2020. The International Application was published in German on Jun. 23, 2022 as WO 2022/127956 under PCT Article 21(2).

### FIELD

The present disclosure relates to methods and to a kit for identifying and/or for obtaining an active substance for the alleviation, treatment and therapy of familial amyotrophic lateral scleroses (ALS), in particular with mutations in SOD1. The disclosure further relates to the use of an active substance for the treatment of ALS, in particular with mutations in SOD1.

### BACKGROUND

Amyotrophic lateral sclerosis is a progressive and fatal neurodegenerative disease associated with the loss of motor neurons. This results in symptoms that include general muscle weakness causing difficulties in moving, breathing, eating and speaking. It affects about 5 in 100,000 individuals, and 80% of affected patients die within 2-5 years after an initial diagnosis. About 20% of all cases of ALS are due to gene mutations, and about 10% of these are due to mutations in the gene for superoxide dismutase 1 (SOD1). In ALS patients with mutations in SOD1, neuronal deposits of mutated and misfolded SOD1 protein are found in motor neurons. Deposits of oligomers and aggregated SOD1 proteins are toxic to motor neurons and lead to their death. Progressive propagation of paralysis in the body is associated with a prion-like multiplication and propagation of toxic SOD1 oligomers and aggregates in the body, where misfolded oligomers/aggregates of mutated SOD1 protein are transferred from one neuron to the next and induce misfolding and subsequent aggregation of native, mutated SOD1 in the recipient cell so that ever greater amounts of toxic SOD1 species are formed. The way in which toxic SOD1 aggregates are transferred between neurons is not fully understood.

There is no cure for ALS, but there are treatments which help to get the disease under control. There are also medicaments for alleviating specific symptoms of the disease, and a number of experimental treatments which are in the development phase. The U.S. Food and Drug Administration (FDA) has approved four treatments that specifically aim to slow the progression of ALS: Rilutek (riluzole tablets), Tiglutik (riluzole suspension), Exservan (riluzole film for ingestion) and Radicava (edaravone). Canada, Australia and Europe have also approved Rilutek, and Tiglutik was approved in the UK under the name Teglutik. Exservan and Radicava are still being investigated in many countries. In Japan, China, the USA and Switzerland, edaravone is approved as a medicament for the treatment of ALS.

None of these treatments can reverse the damage caused by ALS, but researchers believe that they could increase the patient's life expectancy. Physicians often prescribe them in combination with symptomatic treatments in order to improve the patient's quality of life. Although riluzole has been associated with a short survival advantage of 2-3 months, which corresponds to a 9% increase in the 1-year survival rate, the subsequent introduction of riluzole for the treatment of ALS was perhaps evidence of desperation for therapeutic options in the face of this devastatingly progressive disease. In spite of increasing scientific discussion on this topic, the mechanism of therapeutic benefit of riluzole is undetermined. Several pathways have been postulated, ranging from the central anti-glutaminergic modulation of excitotoxic pathways, mitochondrial function and changes in fat metabolism, and on to peripheral axonal effects on persistent sodium channel function and potentiation of calcium-dependent potassium streams.

The mechanism by which Radicava could be effective for ALS is also unknown. It is known that the medicament is an antioxidant, and it is believed that oxidative stress is part of the process that kills neurons in humans with ALS. In clinical studies, some individuals administered Radicava showed a significantly lower decrease in bodily function compared to placebo, measured by the ALS Functional Rating Scale-Revised (ALSFRS-R), a validated evaluation instrument to monitor the progression of disability in patients with ALS. The approval request for Radicava/edaravone at the European Medicines Agency (EMA) was withdrawn by the pharmaceutical company Mitsubishi Tanabe Pharma Corporation (MTPC) after the authority had recommended the rejection of the approval request due to the term of the submitted studies and, according to the authority, insufficient proof of extended survival.

In summary, it can be said that the active substances approved for treatment have no known mechanism of action, and, if at all, lead only to a very slight improvement in the quality of life or to prolongation of life.

### SUMMARY

Provided herein is a method for identifying and/or for obtaining active substances for the alleviation or treatment of ALS, comprising the following steps: a. providing a composition containing cells having the cell surface receptors syndecan-3 and/or syndecan-1, b. contacting the composition from step a) with SOD1 aggregates, c. contacting the composition from step a) with the test substance to be screened, it being possible for step b) and step c) to be performed simultaneously or for step c) to be performed before step b), and d. determining the uptake of the SOD1 aggregates into the cells. Medicaments comprising pentosan polysulfate sodium (PPS) or compounds from the group of heparins or heparin-like compounds, and methods of using the same, are also provided.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1: polyacrylamide gel electrophoresis of non-aggregated and aggregated wild-type SOD1 and mutated A4V and G93A SOD1, both with and without fluorescent labeling,

FIG. 2: flow cytometric determination of uptake of aggregated and non-aggregated SOD1 by wild-type neuro-2A (FIG. 2A), A4V mutant-type (FIG. 2B) and G93A mutant-type (FIG. 2C) cells as a function of incubation time,

FIG. 2D: confocal fluorescence microscopy image of neuro-2a cells after 24 hours of incubation with wild-type SOD1 aggregates,

FIG. 3: flow cytometric determination of the uptake of fluorescently labeled SOD1 aggregates into neuro-2a cells under the influence of various endocytosis inhibitors,

FIG. 4: flow cytometric determination of the uptake of SOD1 aggregates into the wild-type neuro-2a, A4V mutant-type and G93A mutant-type cells after incubation with sodium chlorate, heparin, heparinase I and III, and chondroitinase,

FIG. 5: flow cytometric determination of the uptake of wild-type and mutated SOD1 aggregates into neuro-2a cells after treatment with PPS in increasing concentrations,

FIG. 6A: flow cytometric determination of the uptake of SOD1 aggregates into neuro-2a cells with and without treatment with phosphoinositide phospholipase C,

FIG. 6B: expression of the prion protein with and without treatment with phosphoinositide phospholipase C (PI-PLC),

FIG. 6C: flow cytometric determination of the uptake of aggregated and non-aggregated SOD1 by wild-type neuro-2A cells and by neuro-2A cells without expression of Sdc1 and/or Sdc3,

FIG. 7A: flow cytometric determination of the uptake of fluorescently labeled SOD1 aggregates into primary cortical neurons of wild-type mice after incubation with heparin, heparinase I and III, and chondroitinase,

FIG. 7B: flow cytometric determination of the uptake of fluorescently labeled SOD1 aggregates into primary cortical neurons of Sdc1-knockout mice (Sdc1^{-/-}) compared to wild-type mice,

FIG. 7C: flow cytometric determination of the uptake of fluorescently labeled SOD1 aggregates into primary cortical neurons of Sdc3-knockout mice (Sdc3^{-/-}) compared to wild-type mice; and

FIG. 7D: flow cytometric determination of the uptake of fluorescently labeled SOD1 aggregates into primary cortical neurons of wild-type mice, Sdc1-knockout mice (Sdc1^{-/-}) and Sdc3-knockout mice (Sdc3^{-/-}) over a period of 6 h.

### DETAILED DESCRIPTION

In an embodiment the invention provides a method and a kit with which active substances for the alleviation or treatment of amyotrophic lateral sclerosis, hereinafter referred to as ALS, can be identified. The use of an active substance for the treatment of ALS is provided by an embodiment.

The subject matter of the invention includes a method for identifying and/or for obtaining active substances for the alleviation or treatment of ALS which, in particular, have the effect of stopping propagation of toxic SOD1 aggregates between neurons in organisms, in particular in the human body, and thus inhibiting progressive paralysis in the body of ALS patients with mutations in SOD1.

In the context of the invention, it has been found that two specific cell surface receptors, syndecan-3 and syndecan-1, which belong to the heparan sulfate proteoglycans (HSPG) expressed on the cell surface of neurons, bind superoxide dismutase 1 aggregates (hereinafter referred to as SOD1 aggregates) and mediate their uptake into neurons. This uptake causes and accelerates the previously described propagation of toxic SOD1 aggregates between neurons in organisms.

The method according to embodiments of the invention is characterized in that these aforementioned cell surface receptors to which these toxic SOD1 aggregates bind, in particular syndecan-3 and/or syndecan-1, are to be used as targets for identifying and/or for obtaining active substances for the treatment of ALS, wherein these active substances are to be investigated in particular for their ability to inhibit a binding of SOD1 aggregates to syndecan-3 and/or syndecan-1 and the uptake of these SOD1 aggregates into neurons, and thus to enable the alleviation and treatment of ALS.

The method according to embodiments of the invention for identifying and/or for obtaining active substances for the alleviation or treatment of ALS comprises the following steps:
a. providing a composition containing cells having the cell surface receptors syndecan-3 and/or syndecan-1,
b. contacting the composition from step a) with SOD1 aggregates,
c. contacting the composition from step a) with the test substance to be screened, it being possible for step c) and step b) to be performed simultaneously or for step c) to be performed before step b),
d. determining the uptake of the SOD1 aggregates into the cells.

In an advantageous embodiment of the method, after step d), an additional method step e) is carried out in which the results of the uptake of the SOD1 aggregates into the cells are compared after step b) with a reference value, without the addition of the test substance (also referred to synonymously as the active substance). In this way, it is advantageously possible on the one hand to verify whether the method approach functions in principle, and the cells are in principle capable of taking up the SOD1 aggregates into the cells without the addition of the test substance. On the other hand, it is possible, for example, to determine the effect of the test substance in terms of its actual effect or in terms of its specific duration of efficacy and dose qualitatively and quantitatively, in comparison with the reference value of the uptake of the SOD1 aggregates into the cells without the addition of the test substance.

Thus, if no uptake, or a reduced uptake compared to the reference value, of SOD1 aggregates into the cells is detected in the presence of the active substance to be screened, an inhibiting effect for this active substance can be demonstrated and this active substance can then be selected either for further studies in the treatment of ALS or directly as an active substance candidate for clinical studies.

For example, neurons, neuronal cell lines or other cells/cell lines that have been modified such that they express the cell surface receptors syndecan-3 and/or syndecan-1 can be used as the cells.

In one embodiment of the method, it may be advantageous to use cells which express both syndecan-3 and syndecan-1, since the inhibitory effect of the active substance for the uptake of SOD1 aggregates can thereby be investigated simultaneously via both syndecans. However, it is of course also possible to use cells that express either syndecan-3 or syndecan-1.

Any SOD1 aggregates which are known for triggering ALS in humans or also in animal models can be used as SOD1 aggregates. These can be both wild-type SOD1 aggregates and mutated SOD1 aggregates that are known to trigger ALS. SOD1 aggregates with the familial A4V or G93A mutation have also proven advantageous.

It is advantageous to use SOD1 aggregates which are capable of emitting a specific signal which can be used not only as proof for the binding of the SOD1 aggregates to the syndecans but also as proof for the determination of the uptake of the SOD1 aggregates into the cells. For example, fluorescence signals that can detect the uptake of SOD1 aggregates into the cells by means of optical detection methods, preferably flow cytometry or microscopic methods, have proven suitable in this context. For this purpose, the SOD1 aggregates are preferably labeled with a fluorescent dye suitable for detection, such as DyLight488 or further fluorescent dyes known to the person skilled in the art and suitable for use.

In principle, all methods known to the person skilled in the art for the detection of protein uptake and the quantification of these proteins in cells are suitable for determining the uptake of the SOD1 aggregates into the cells. In this respect, for example, but not limited thereto, immunoassays, preferably ELISA or MA, or spectroscopic methods, such as mass spectrometry, mass spectroscopy, NMR, flow cytometry, microfluidic flow cytometry, combinations of the aforementioned methods, or any microscopic detection methods can be mentioned.

The invention moreover relates to a kit or a composition for conducting the method according to embodiments of the invention, containing at least the cells mentioned above in detail and having the cell surface receptors syndecan-3 and/or syndecan-1, and also SOD1 aggregates, which likewise have been described in detail above.

Another subject matter of the invention is the use of an active substance for the treatment of ALS, which inhibits the binding of SOD1 aggregates to the cell surface receptors syndecan-1 and syndecan-3, and thus stop the propagation of toxic SOD1 aggregates between neurons in the body, thereby being able to inhibit progressive paralysis in the body of ALS patients with mutations in SOD1.

For the use of an active substance for the treatment of ALS, the compound pentosan polysulfate sodium (PPS) and compounds from the group of heparins or heparin-like compounds, such as heparin (natural), certoparin, dalteparin, enoxaparin, nadroparin, reviparin, tinzaparin, danaproid, deligoparin, fucoidan, have proven suitable. By using these aforementioned compounds, the prion-like propagation of toxic SOD1 aggregates between neurons in the body can advantageously be inhibited.

PPS has previously been used as a medicament for the treatment of interstitial cystitis, of thromboses, and osteoarthritis. Further use is the treatment of osteoarthritis in dogs and horses in veterinary medicine.

In accordance with the invention, pentosan polysulfate sodium (PPS) or compounds from the group of heparins or heparin-like compounds can therefore also be used for the preparation of an inhibitor for the uptake of SOD1 aggregates into cells of an organism.

A further subject matter of the invention is pentosan polysulfate sodium (PPS) or compounds from the group of heparins or heparin-like compounds as medicaments in the treatment of ALS.

Furthermore, medicaments containing pentosan polysulfate sodium (PPS) or compounds from the group of heparins or heparin-like compounds and a pharmaceutically acceptable excipient are the subject matter of the invention. All pharmaceutical excipients known according to the prior art are suitable here.

The use of pentosan polysulfate sodium (PPS) or compounds from the group of heparins or heparin-like compounds in the preparation of a medicament for the treatment of ALS is likewise a subject matter of the invention. In one possible embodiment of the invention, pentosan polysulfate sodium (PPS) or compounds from the group of heparins or heparin-like compounds are provided, which comprise(s) a pharmaceutically acceptable diluent and a therapeutically effective amount of this substance or these compounds.

Furthermore, embodiments of the invention also relate to the use of pentosan polysulfate sodium (PPS) or compounds from the group of heparins or heparin-like compounds in the preparation of an inhibitor for the uptake of SOD1 aggregates into cells of an organism.

Embodiments of the invention also relate to methods for inhibiting the uptake of SOD1 aggregates into neurons of an organism, in which methods pentosan polysulfate sodium (PPS) or compounds from the group of heparins or heparin-like compounds, or a pharmaceutical formulation of this substance or compounds, is/are used. In this method, an organism is administered pentosan polysulfate sodium (PPS) or compounds from the group of heparins or heparin-like compounds, or a pharmaceutical formulation of this/these medicament(s).

The administration to the organism is preferably a therapeutically effective amount. Preferably, the organism is a human or an animal model. The aforementioned compounds can be administered to the organism, for example, orally, intravenously or by inhalation.

The use of a composition for the treatment and therapy of ALS containing at least the compound pentosan polysulfate sodium (PPS) or compounds from the group of heparins or heparin-like compounds is likewise a subject matter of embodiments of the invention.

The invention is explained in more detail below with reference to experimental exemplary embodiments, which demonstrate the mode of action of syndecan-3 and syndecan-1 and of PPS, and to several figures which reproduce the experimental data. The invention is not limited to the exemplary embodiments described here by way of example. All described and/or depicted features can manifest individually or in combination in different embodiments. The features of the different embodiments of this invention and their respective advantages are disclosed when reading the exemplary embodiments described below in connection with the figures.

### 1) Expression, Labeling, and Aggregation of SOD1

For the experimental exemplary embodiments, expressed and purified recombinant wild-type (wt) SOD1 and mutated human SOD1 aggregates with the familial A4V or G93A mutation from E. coli were used.

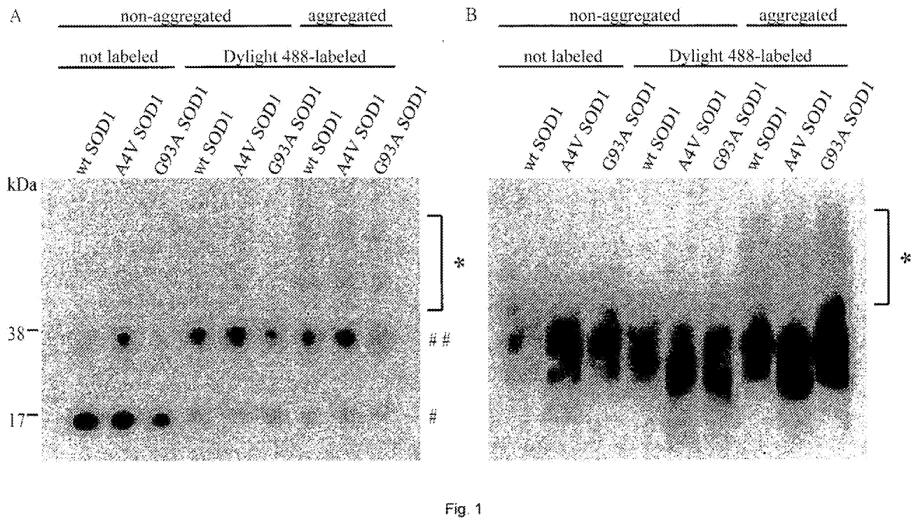

All three SOD1 aggregate variants (wt SOD1, A4V SOD1, G93A SOD1) were fluorescently labeled with DyLight 488. SOD1 aggregates were prepared by treatment with trifluoroethanol. All three non-aggregated and aggregated SOD1 variants were analyzed using polyacrylamide gel electrophoresis under denaturing conditions (FIG. 1A), followed by immunoblotting with an antibody against human SOD1. The results showed that the monomeric SOD1 was about 16 kDa and had naturally formed dimers of about 32 kDa. The fluorescence labeling of SOD1 with DyLight 488 stabilized the dimeric form of all three SOD1 variants under these electrophoresis conditions.

Upon aggregation, fluorescently labeled SOD1 formed high-molecular-weight species which could be detected as a continuous smear (in FIGS. 1A and 1B of the gel band region marked with * within square brackets) with lower electrophoretic mobility. Immunoblotting of gels carried out under non-denaturing conditions with the same human SOD1 antibody confirmed that aggregation of SOD1 resulted in high-molecular-weight species which exhibited lower electrophoretic mobility than non-aggregated SOD1 (FIG. 1B).

### 2) Detection of Improved Uptake of SOD1 Aggregates by Cells

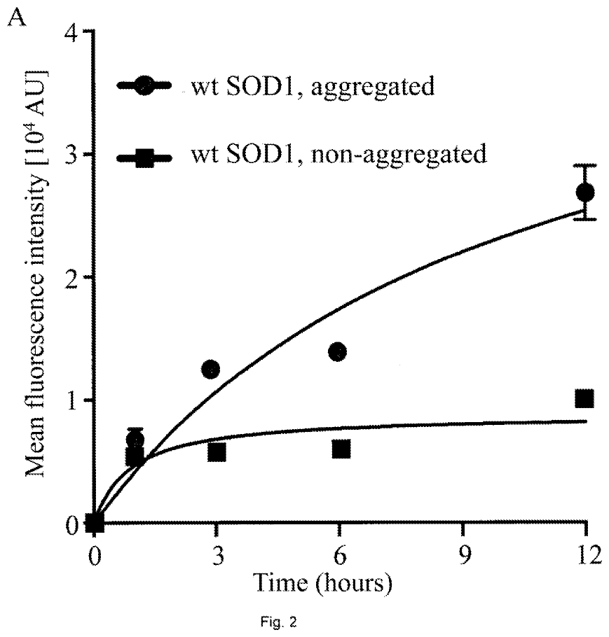

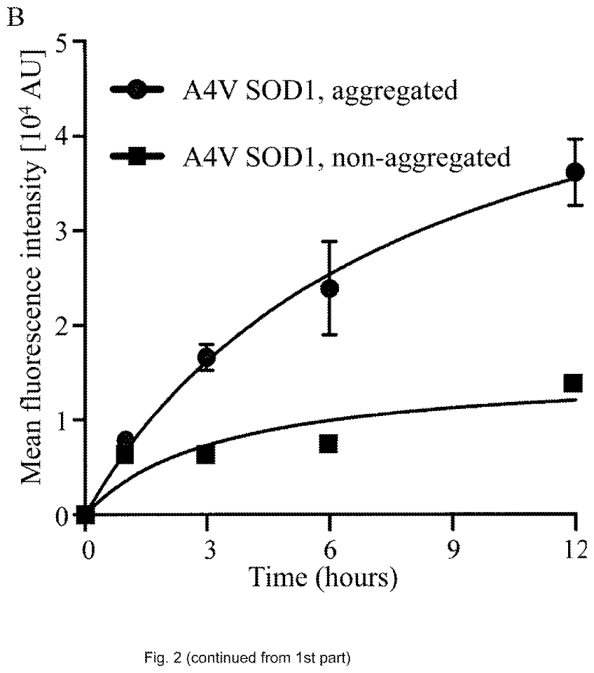

In order to examine the uptake of SOD1 by neuro-2a cells as a function of time, wild-type neuro-2a cells (FIG. 2A) as well as the A4V (FIG. 2B) and the G93A mutants (FIG. 2C) were incubated with fluorescently labeled, non-aggregated or aggregated SOD1. Subsequently, the amount of uptake of aggregated or non-aggregated SOD1 was ascertained by means of flow cytometry after 1, 3, 6 and 12 hours. Neuro-2a cells were able to internalize both non-aggregated and aggregated SOD1. Incubations of more than one hour showed that the uptake of aggregated SOD1 in contrast to that of non-aggregated SOD1 was significantly increased, which was apparent due to consistently higher levels of internalized SOD1 aggregates. Interestingly, neuro-2a cells internalized wild-type SOD1 aggregates just as well as aggregates formed by the two mutated SOD1 variants, suggesting that a mutation in SOD1 is not essential for their cellular uptake. Selective treatment of the cells with trypsin for 30 minutes after prior treatment with fluorescently labeled SOD1 did not significantly alter the fluorescence signal. (Data not shown) indicating that the measured signal originated from SOD1 proteins that the cells had internalized, and not from SOD1 proteins bound to the outer cell membrane. Confocal fluorescence microscopy of neuro-2a cells treated with wild-type SOD1 aggregates (light gray) for 24 hours also showed that cells (with cell nuclei stained dark gray with Hoechst) had internalized the SOD1 aggregates (FIG. 2D).

3) Macropinocytosis of SOD1 Aggregates into Cells

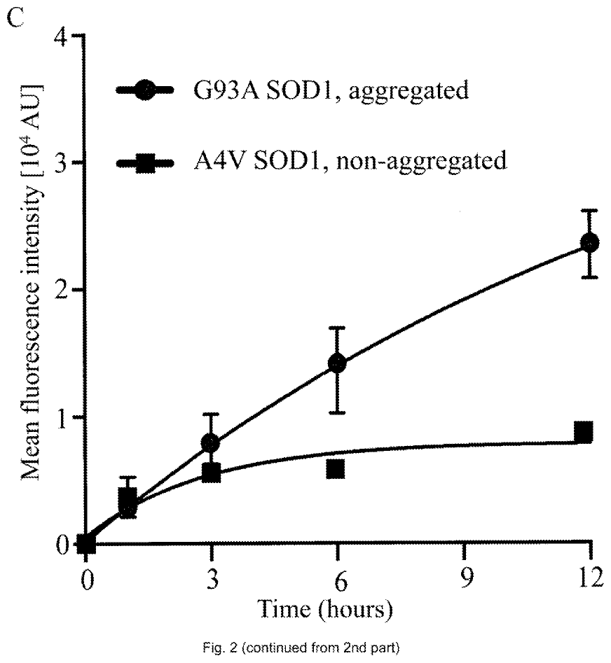

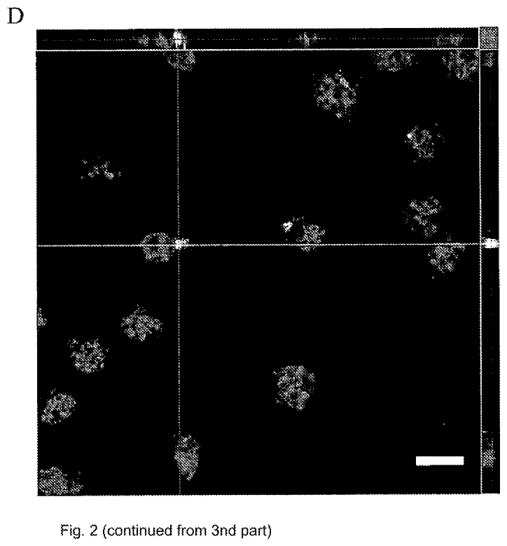

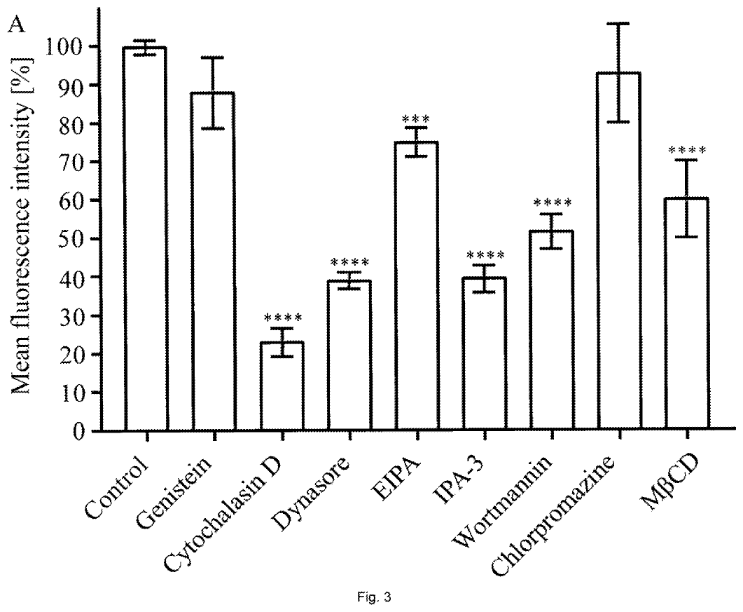

The exact mechanism through which SOD1 aggregates enter the cells has hitherto been unknown. Endocytosis of extracellular proteins can occur via different pathways. In order to analyze by which mechanism SOD1 aggregates of neuro-2a cells are internalized, cells were treated with various endocytosis inhibitors and the uptake of fluorescently labeled SOD1 aggregates was measured by flow cytometry (FIG. 3).

Chlorpromazine inhibits clathrin-mediated endocytosis by preventing the formation of clathrin lattices on the cell surface. Genistein is an inhibitor of protein tyrosine kinases and is used to inhibit caveola-mediated endocytosis. Neither the chloropromazine treatment nor the genistein treatment inhibited the uptake of SOD1 aggregates significantly, suggesting that uptake was neither dependent on caveolin nor mediated by clathrin.

In contrast, the treatment with cytochalasin D, an inhibitor of actin polymerization, significantly inhibited the uptake, suggesting an actin-dependent process. Treatment with methyl-β-cyclodextrin (MβCD), a cholesterol-reducing agent, also significantly reduced the uptake of SOD1 aggregates, suggesting that the uptake is dependent on lipid rafts. Treatment with EIPA (5-N-ethyl-N-isopropyl amiloride), an inhibitor of Na⁺/H⁺ exchangers, wortmannin, an inhibitor of phosphoinositide-3-kinase, and IPA-3 (2,2′-dihydroxy-1,1′-dinaphthyl disulfide), an inhibitor of p21-activated kinase 1 (PAK1), reduced the uptake of SOD1 aggregates significantly, suggesting that macropinocytosis was involved. In the cell assay used, dynasore inhibited the uptake of SOD1 aggregates. Dynasore was originally identified as an inhibitor of dynamin, a GTPase protein, which is essential for membrane cleavage during clathrin-mediated endocytosis in eukaryotic cells. However, dynasore also reduces labile cholesterol in the plasma membrane and disrupts lipid raft organization and membrane cleavage independently of dynamin, leading to inhibition of macropinocytosis. In view of the fact that chloropromazine, unlike MβCD, does not inhibit the uptake of SOD1 aggregates, our results provide further evidence for the uptake of SOD1 aggregates by macropinocytosis. The “***” or “****” above the bars indicate the statistical significance of the results. “***” corresponds to a significance with p≤0.001 and “****” corresponds to a significance with p≤0.0001 in comparison to the control.

### 4) Heparan Sulphate Proteoglycans (HSPG) Mediate the Uptake of SOD1 Aggregates

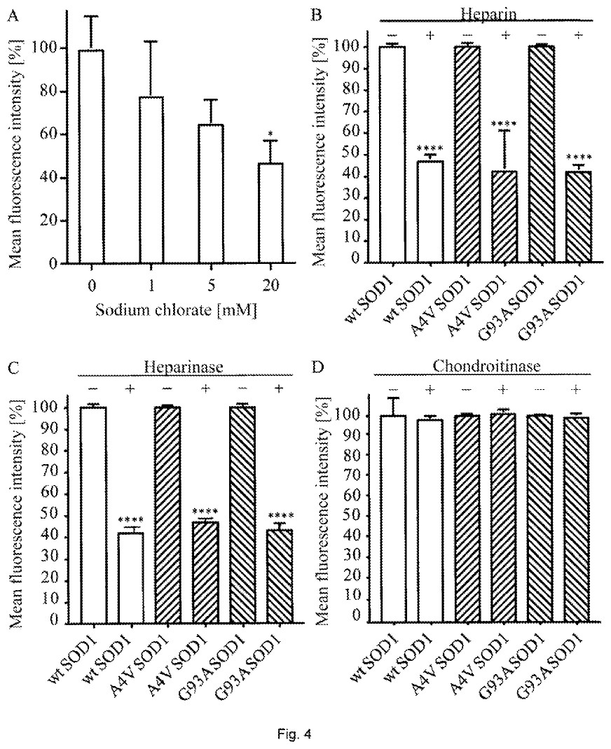

Cell access via macropinocytosis can be induced by the binding of specific viruses, bacteria or protein aggregates, including aggregated tau species, amyloid beta, α-synuclein, and infectious prion proteins to HSPG bound to the cell membrane. In order to determine whether cells take up SOD1 aggregates via HSPG, wild-type neuro-2a cells were treated for 48 h with sodium chlorate, which arrests the proper sulfation of HSPG, before the cells were incubated with SOD1 aggregates for 12 hours and their uptake was measured by flow cytometry. Treatment with increasing sodium chlorate concentrations reduced the uptake of SOD1 aggregates in a dose-dependent manner (FIG. 4A).

Next, heparin, a naturally occurring glycosaminoglycan, structurally related to heparan sulfate, was used to competitively inhibit the binding of SOD1 aggregates to HSPG. For this purpose, the SOD1 aggregates were treated at 4° C. with 1 mg/mL of heparin for 6 hours. Subsequently, the neuro-2a cells were incubated with these pre-treated SOD1 aggregates for 6 hours. The results show that heparin inhibited the uptake of both wild-type and mutated SOD1 aggregates by neuro-2a cells (FIG. 4B), a process that was dose-dependent (data not shown). Similarly, the 2-hour treatment of neuro-2a cells with a mixture of heparinase I and III (2 U/mL), two enzymes that recognize heparan sulfates on HSPG as their primary target, resulted in reduced uptake of both wild-type and mutated SOD1 aggregates by neuro-2a cells when measured by flow cytometry after 6 hours (FIG. 4C). In contrast, the 2-hour treatment of neuro-2a cells with chondroitinase AC (2 U/mL), an enzyme that specifically cleaves chondroitin sulfate, another glycosaminoglycan that is found as a carbohydrate moiety on some proteoglycans, did not significantly affect the uptake of SOD1 aggregates (FIG. 4D). The results indicate that SOD1 aggregates are specifically absorbed by HSPG since the uptake could be chemically inhibited with sodium chlorate, competitively with heparin, and enzymatically with heparinases, but not with chondroitinase. The “***” or “****” above the bars indicate the statistical significance of the results. “***” corresponds to a significance with p≤0.001 and “****” corresponds to a significance with p≤0.0001 in comparison to the control.

### 5) Pentosan Polysulfate Sodium (PPS) Inhibits Uptake of SOD1 Aggregates

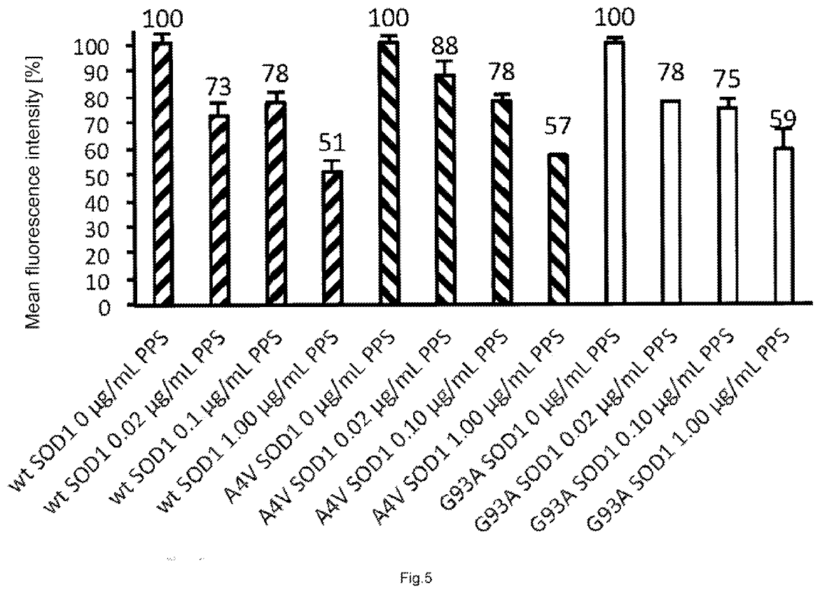

PPS is part of the most effective medicaments used in experimental models of prion diseases. PPS is known for extending the survival of infected animals after intracerebroventricular perfusion in a dose-dependent manner. PPS is also used for the experimental treatment of human prion diseases in the context of clinical studies. PPS will compete with endogenous HSPG on the cell surface as a co-receptor for infectious prions. Treatment of neuro-2a cells with increasing PPS concentrations competitively inhibited the cellular uptake of fluorescently labeled wild-type and mutated SOD1 aggregates at non-cytotoxic concentrations (FIG. 5).

6) Evidence for the Syndecan-1- and Syndecan-3-Mediated Uptake of SOD1 Aggregates into Neuro-2a Cells

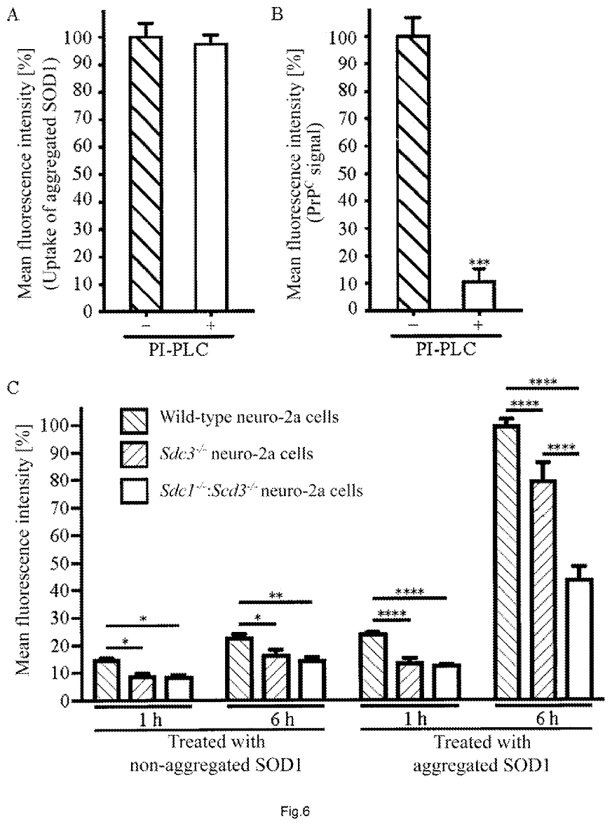

Glypicans 1-6 are a family of HSPG attached to the outer cell membrane via a GPI anchor. In order to demonstrate whether glypicans can mediate the uptake of SOD1 aggregates, neuro-2a cells were treated with phosphoinositide phospholipase C (PI-PLC), which releases GPI-anchored proteins from the outer cell membrane. Treatment of neuro-2a cells with phosphoinositide phospholipase C did not reduce the uptake of SOD1 aggregates into neuro-2a cells, as demonstrated by flow cytometry (FIG. 6A).

The expression of the prion protein, a further GPI-anchored protein, was reduced by more than 90% after PI-PLC treatment (FIG. 6B), showing that PI-PLC treatment has effectively led to the release of GPI-anchored proteins.

These data demonstrate that glypicans do not mediate the uptake of SOD1 aggregates.

Syndecans 1-4 are a family of transmembrane HSPG. Since syndecan-3 (Sdc3) and syndecan-1 (Sdc1) have a very high sequence homology and are expressed in neurons, we used CRISPR/Cas9 to specifically generate neuro-2a cells that do not have expression of Sdc3 or of Sdc1, or have no expression of Sdc1 and Sdc3. As previously shown for wild-type neuro2 cells (FIG. 2), the uptake of aggregated SOD1 was higher than that of non-aggregated SOD1, not only in wild-type cells but also in cells lacking expression of Sdc3 or of Sdc1 and Sdc3 (FIG. 6C). In cells in which both the Sdc1 and the Sdc3 expression were missing (white bar), the uptake of monomeric and aggregated SOD1 was significantly reduced compared to the wild-type cells by almost 60%. (FIG. 6C). The “***” or “****” above the bars indicate the statistical significance of the results. “***” corresponds to a significance with p≤0.001 and “****” corresponds to a significance with p≤0.0001 in comparison to the control.

7) Syndecan-1- and Syndecan-3-Mediated Uptake of SOD1 Aggregates into Primary Neurons

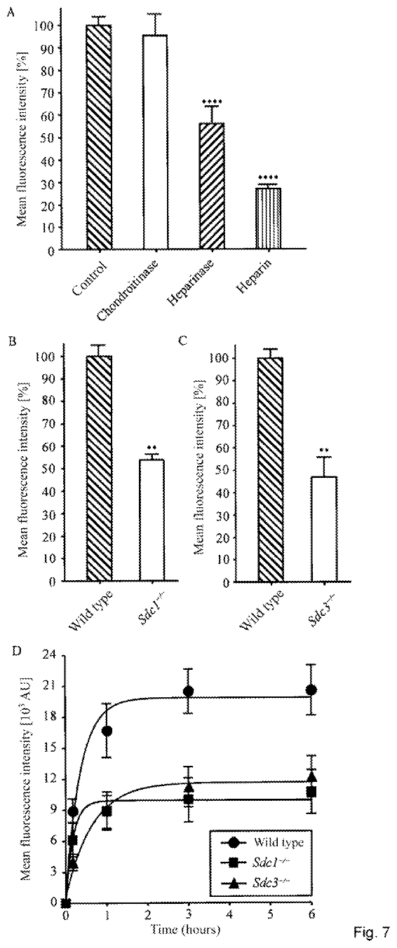

In order to investigate whether the results in neuro-2a cells can be expanded to primary cortical neurons, isolated, cultured and treated primary cortical neurons of wild-type mice were treated with heparinase and chondroitinase as previously for neuro-2a cells, before they were exposed to DyLight 488-labeled SOD1 aggregates (FIG. 7A). Likewise, the uptake of SOD1 aggregates after blocking with heparin was tested, as previously carried out for neuro-2a cells (FIG. 7A). The uptake of SOD1 aggregates was again quantified by flow cytometry. The results show that the enzymatic treatment with heparinase and competitive blocking with heparin, but not the enzymatic treatment with chondroitinase, inhibits uptake of SOD1 aggregates into primary cortical neurons, in the same way as observed in neuro-2a cells (FIG. 4). The “***” or “****” above the bars indicate the statistical significance of the results. “***” corresponds to a significance with p≤0.001 and “****” corresponds to a significance with p≤0.0001 in comparison to the control.

Next, isolated, cultured, primary cortical neurons of Sdc1-knockout (Sdc1^{-/-}) and Sdc3-knockout (Sdc3^{-/-}) mice were treated with fluorescently labeled SOD1 aggregates. The flow cytometric measurement showed that the uptake of fluorescently labeled SOD1 aggregates into primary cortical neurons without Sdc1^{-/-} (FIG. 7B) or Sdc3^{-/-} (FIG. 7C) was reduced by 50% compared to primary cortical neurons of wild-type mice. Real-time imaging of the uptake of SOD1 aggregates into cultured primary cortical neurons of Sdc1^{-/-} mice or Sdc3^{-/-} mice over a period of 6 h also showed that uptake of SOD1 aggregates compared to cultured primary cortical neurons of wild-type mice was reduced by about half (FIG. 7D).

The data demonstrate that syndecan-1 and syndecan-3 play a crucial role in the uptake of SOD1 aggregates into neuronal cells, and suggest that both receptors can mediate cell-to-cell transfer of pathologic SOD1 aggregates in ALS, which could lead to a spread of the disease along the motor axis.

Although the invention has been illustrated and described in detail in the experimental examples and the preceding description, these experimental data and descriptions should be regarded as illustrative or exemplary and not as limiting. It should be understood that within the scope of the following claims, changes and modifications may be made using conventional skills. In particular, the present invention comprises further embodiments with any combination of features of the various embodiments described above and below.

While the invention has been described and depicted in detail in the preceding part of the application on the basis of special exemplary embodiments, this description and the figures are intended merely to apply as an example without thereby acting in a limiting manner. It can be assumed that within the scope of their technical knowledge, a person skilled in the art would and could carry out further changes and modifications to the following claims which are covered by the scope of protection of the claims. In particular, further embodiments with any type of combinations of the mentioned features of individual exemplary embodiments are included in the scope of the invention.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A method for identifying and/or for obtaining active substances for the alleviation or treatment of ALS, comprising the following steps:
a. providing a composition containing cells having the cell surface receptors syndecan-3 and/or syndecan-1,
b. contacting the composition from step a) with SOD1 aggregates,
c. contacting the composition from step a) with the test substance to be screened, it being possible for step b) and step c) to be performed simultaneously or for step c) to be performed before step b), and
d. determining the uptake of the SOD1 aggregates into the cells.

2. The method according to claim 1, wherein in step e), the results of the uptake of the SOD1 aggregates into the cells are compared after step c) with a reference value without the addition of the test substance.

3. The method according to claim 1, wherein neurons, neuronal cell lines, or cells/cell lines that have been modified such that they express the cell surface receptors syndecan-3 and/or syndecan-1 are used as cells.

4. The method according to claim 1, wherein SOD1 aggregates that are known to trigger ALS in humans or in animal models are used.

5. The method according to claim 1, wherein wild-type SOD1 aggregates or mutated SOD1 aggregates are used.

6. The method according to claim 1, characterized in that SOD1 aggregates with the familial A4V or G93A mutation are used.

7. The method according to claim 1, wherein SOD1 aggregates that are labeled or are capable of emitting a specific signal are used.

8. The method according to claim 1, wherein SOD1 aggregates that are labeled with a fluorescent dye are used.

9. The method according to claim 1, wherein the determination of the uptake of the SOD1 aggregates into the cells according to step d) is carried out in cells by means of protein analysis methods.

10. The method according to claim 1, wherein the determination of the uptake of the SOD1 aggregates into the cells according to step d) is carried out by means of optical methods.

11. The method according to claim 1, wherein the determination of the uptake of the SOD1 aggregates into the cells according to step d) is carried out by means of flow cytometry, immunological detection methods, spectrometric methods, or microscopic methods.

12. A kit or composition for conducting a method according to claim 1, containing comprising at least cells expressing the cell surface receptors syndecan-3 and/or syndecan-1, and also SOD1 aggregates.

13. (canceled)

14. (canceled)

15. A medicament comprising pentosan polysulfate sodium (PPS) or compounds from the group of heparins or heparin-like compounds, and also a pharmaceutically acceptable excipient.

16. (canceled)

17. A method of inhibiting uptake of SOD1 aggregates into cells of an organism comprising administering pentosan polysulfate sodium (PPS) or compounds from the group of heparins or heparin-like compounds to the organism.

18. A method of treatment and/or therapy of ALS comprising administering the medicament of claim 15 to a subject.

19. The method of claim 17, wherein the organism is a human.

20. The method of claim 18, wherein the subject is a human.
